(19) United States
(12) Patent Application Publication
(10) Pub. No.: US 2019/0228837 A1
(43) Pub. Date: Jul. 25, 2019

(54) Rapid Genome Identification and Surveillance Systems

(71) Applicants: The Regents of the University of California, Oakland, CA (US); Shriners Hospitals for Children, Tampa, CA (US)

(72) Inventor: Kiho Cho, Davis, CA (US)

(21) Appl. No.: 16/303,899

(22) PCT Filed: May 23, 2017
(86) PCT No.: PCT/US17/34021

§ 371 Date: Nov. 21, 2018

Related U.S. Application Data
(60) Provisional application No. 62/340,722, filed on May 24, 2016.

(51) Int. Cl. G16B 30/00; C12Q 1/686; C12Q 1/6806; G01N 27/447; G16B 50/00; G16B 20/20; G16B 20/50

(52) U.S. Cl. CPC G16B 30/00; C12Q 1/686; C12Q 1/6806; G16B 20/50; G16B 50/00; G16B 20/20; G01N 27/447

(57) ABSTRACT

This disclosure relates to methods of creating dideoxynucleotide termination frequency (DTF) normalized landscape matrices and time/intensity (TI) normalized landscape matrices, and various applications of the normalized landscape matrices for genomic surveillance, identification, and monitoring of humans, animals, plants, cells and bacteria.

### TECHNICAL FIELD

This disclosure relates to genome identification and surveillance systems.

### BACKGROUND

The vast majority of core concepts and relevant methodologies for modern studies of both normal and disease biology are stringently tethered to the function and polymorphism of “conventional” genes. Conventional gene sequences are reported to be shared among a wide range of species, ranging from rodents to humans (˜85% between humans and mice). It is estimated that the sum of all conventional gene sequences (exons) represents ˜1.2% of the reference human and mouse genomes that have not been completely sequenced yet.

Currently, many genome identification/surveillance methods for humans, animals, and plants primarily focus on polymorphisms in small sets of conventional gene and/or microsatellite sequences. Many of these methods are not cost-effective, and the limited and low-resolution information obtained from polymorphism analyses of individual conventional genes and/or a biased small set of microsatellite polymorphisms are often inadequate for genome identification/surveillance purposes.

### SUMMARY

This disclosure relates to genome identification and surveillance systems.

In one aspect, the present disclosure provides methods of creating a dideoxynucleotide termination frequency (DTF) normalized landscape matrix. The methods include the steps of providing a plurality of amplicons having different genomic elements/sequences, optionally wherein the amplicons are provided by digestion and/or ligation of genomic DNA prior to PCR amplification; performing a dideoxynucleotide termination sequencing reaction on a reaction mixture having the plurality of amplicons having different genomic elements/sequences, using a primer that binds to the plurality of amplicons at a plurality of different binding sites; obtaining an intensity of fluorescence for each type of nucleotide (A, T, G, C) at each individual nucleotide position in the heterogeneous population of amplicons (i.e., downstream of the primer binding sites); normalizing the intensity of fluorescence of each nucleotide type at each individual nucleotide positions; and creating a matrix of the normalized intensity of fluorescence for each type of nucleotide at each individual nucleotide position; thereby creating a DTF normalized landscape matrix.

In another aspect, the present disclosure relates to methods of creating a time/intensity (TI) normalized landscape matrix. The methods include the steps of providing a plurality of amplicons having different genomic elements/sequences, optionally wherein the amplicons are provided by digestion and/or ligation of genomic DNA prior to PCR amplification; performing capillary electrophoresis (CE) analysis of the plurality of amplicons having different sequences, optionally after restriction digestion; obtaining time (second)/size-intensity (mV) values over a specified time period from the CE analysis; and normalizing the amplicon/fragment intensity at each time point/size by dividing the intensity values by a baseline value, thereby creating a normalized time/size-intensity landscape matrix (TI-NLM) for each sample.

In some embodiments, the plurality of amplicons is obtained using one or more PCR reactions, wherein the PCR reactions are configured to amplify heterogeneous elements/regions in a genome.

In some embodiments, the plurality of amplicons is obtained using single-multiplex PCR.

In some embodiments, the plurality of amplicons includes repetitive elements, B-cell receptors, T-cell receptors, or protocadherin gene clusters.

The present disclosure also provides methods of determining a genetic identity of a cell, tissue, organ, or organism. The methods include the steps of creating a DTF or TI normalized landscape matrix for the genome of the cell, tissue, organ, or organism, according to the method of claim 1 or 2; determining the distance-correlation between the DTF or TI normalized landscape matrix of a test sample and a DTF or TI normalized landscape matrix of a reference sample, optionally wherein the reference sample has a known genetic identity; and optionally determining whether the distance is less than a reference threshold; thereby determining the genetic identity of a cell, tissue, organ, or organism.

In some embodiments, the cell, tissue, organ, or organism is, or is from, an animal, a plant, a fungus or a bacterium. In some embodiments, the animal is a mammal (e.g., a human), a bird, a fish, or a reptile. In some embodiments, the cell, tissue, organ, or organism is, or is from, a genetically modified animal or a genetically modified plant.

The present disclosure also relates to methods of determining whether a test subject has a disease. The methods include the steps of creating a DTF or TI normalized landscape matrix of the test subject; calculating the distance between the DTF or TI normalized landscape matrix of the test subject and one or more DTF or TI normalized landscape matrices that represent a subject having the disease; and comparing the distance to a reference threshold, and concluding that the test subject has the disease if the distance is less than a reference threshold.

In some embodiments, the disease is cerebral palsy, autism spectrum disorder, ductal carcinoma in situ, breast cancer or an aging-related disorder.

The present disclosure also relates to methods of identifying a genetic risk factor in a test subject. The methods include the steps of creating a DTF or TI normalized landscape matrix of the test subject; calculating the distance between the DTF or TI normalized landscape matrix of the test subject and one or more DTF or TI normalized landscape matrices representing a subject having the genetic risk factor; and comparing the distance to a reference threshold, and identifying the test subject as having the genetic risk factor if the distance is less than a reference threshold.

In some embodiments, the test subject is a fetus or an embryo.

The present disclosure also provides methods of monitoring the genome of a subject. The methods include the steps of creating a DTF or TI normalized landscape matrix for the subject at a first time point; creating a DTF or TI normalized landscape matrix for the subject at a second time point; and calculating the distance between the DTF or TI normalized landscape matrix of the first time point and the DTF or TI normalized landscape matrix of the second time point; thereby monitoring the genome of the subject.

In some embodiments, the subject is receiving a therapy between the first and second time points, e.g., radiation therapy or a chemotherapy.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a flow chart of one exemplary protocol of performing collection of heterogeneous genomic elements, dideoxynucleotide (ddNTP) termination frequencies (DTF) sequencing, and creating DTF normalized landscape matrix (DTF-NLM) for distance/correlation computation among different genomes.

FIGS. 2A-2E are diagrams showing five exemplary applications of the DTF-NLM genome identification and surveillance systems.

FIGS. 3A-3B is a flow chart of one exemplary protocol for creating and analyzing a time/size-intensity normalized landscape matrix (TI-NLM).

FIG. 4 is a diagram showing an exemplary protocol for transforming a pool of heterogeneous RE landscape amplicons from individual microbial genomes to a computable numeric matrix for machine learnable identification and surveillance of microbial species and strains by the RaPIdMicro system.

FIG. 5 is a diagram showing a system summary of some exemplary protocols for genome surveillance technology (GST)-based genomic endogenous retrovirus (ERV) landscaping for authentication and surveillance of cell lines.

FIG. 6 is a diagram showing some exemplary protocols for collection of heterogeneous ERV amplicons, numeric transformation by ddNTP reaction, normalization, and correlation computation for cell line authentication.

FIG. 7 is a diagram showing some exemplary protocols for collection of heterogeneous ERV amplicons, numeric transformation by capillary electrophoresis, normalization, and correlation computation for cell line authentication.

FIG. 8 is a diagram showing some exemplary schemas for the construction of the machine-learnable Genetics Surveillance Systems based on the Rapid Genome Identification and Surveillance technologies for determining identification, diagnostics, and divergence of all life forms (humans, animals, plants, and microbes).

### DETAILED DESCRIPTION

Currently, many genome identification/surveillance methods for humans, animals, and plants primarily focus on polymorphisms in small sets of conventional gene and/or microsatellite sequences. In fact, the results from recent studies demonstrated that the current conventional gene/microsatellite-based protocols provide insufficient data for the correct identification/surveillance of individual genome samples.

Described herein are methods involving protocols, algorithms, and systems that can be used for rapid, cost-efficient, unbiased, tunable, and high-resolution genome identification/surveillance by collecting heterogeneous genomic elements followed by transforming, normalizing, and correlation/distance-computing diverse repetitive elements (RE) landscape data, e.g., dideoxynucleotide (ddNTP) termination frequencies (DTF) normalized landscape matrix and time/size-intensity (TI) normalized landscape matrix. The normalized landscape matrix (NLM) based genome identification/surveillance platform, which utilizes the DTF information or TI information from heterogeneous genomic element clusters, is applicable to a wide range of species and fields by rapidly and cost-effectively presenting new types of precise genomic landscape information.

The normalized landscape matrix (NLM) based genome identification/surveillance systems are built upon the observation that the genomic identity of all life forms, ranging from plants to humans, can be rapidly discerned by pattern computation of a heterogeneous population of REs following transformation and normalization of their DTFs or TIs. The NLM systems are developed to generate rapid, cost-effective, and high-resolution genome identification/surveillance data.

In some embodiments, the genome landscaping systems described herein transform heterogeneous genomic element data, such as repetitive elements (REs: both transposable and non-transposable), derived from an individual's genome into a normalized numeric landscape matrix format by computation of Sanger's dideoxynucleotide termination frequencies (DTFs) at each sequence position. In some embodiments, the DTF data type can be replaced with the raw data (fragment intensity values at individual time points (equivalent to DNA fragment sizes)) embedded in the electropherograms produced by capillary electrophoresis (CE) analyses of heterogeneous genomic elements (e.g., REs). Applying the same work-flow as the DTF-NLM systems, the raw intensity-time data from CE analyses can be normalized before it is subjected to distance/correlation computation for genetic identification and surveillance. Thus, in some embodiments, the genome landscaping systems described herein transform heterogeneous genomic element data, such as repetitive elements (REs: both transposable and non-transposable), derived from an individual's genome into a normalized numeric landscape matrix format by computing time/size-intensity data at a series of time points.

In addition to REs, other heterogeneous genomic elements can be used in the present methods. These heterogeneous genomic elements include, e.g., B-cell receptors (BCRs), T-cell receptors (TCRs), protocadherins, and other clusters of genomic elements.

The NLM landscaping-based genome identification/surveillance can be applied to a wide range of organisms (e.g., humans, animals, and plants, fungi, and bacteria) and fields, such as forensic sciences, animal breeding, plant breeding, pharmacogenomics, monitoring of radiation therapy, cell/tissue typing, diagnostics-marker discovery, genome toxicology, embryo screening, immune surveillance, genotyping of genetically modified/edited cells and organisms, and studies of normal and disease states.

The following highlights some of the unique features and advantages of some embodiments of NLM Genome Identification and Surveillance Systems as described herein:
1. For heterogeneous RE populations, RE target information (RE type, size, sequence, and/or position) can be collected de novo, as RE PCR amplicons are generated for the unbiased identification/surveillance of specific genomes/cells.
2. For heterogeneous B Cell Receptor/T Cell Receptor (BCR/TCR) populations, BCR/TCR target information (segment type, size, sequence, and/or junction combination-position) can be collected de novo, as the BCR/TCR PCR amplicons are generated for the unbiased identification/surveillance of immune cell profiles.
3. For heterogeneous populations of protocadherins and other genomic element clusters, relevant target information (segment type, size, sequence, and/or junction combination-position) can be collected de novo as the relevant PCR amplicons are generated for the unbiased identification/surveillance of neuronal/other cell profiles.
4. Implementation of NLM algorithms and genomic amplicon/fragment collection technologies provides for rapid and cost-efficient genome identification/surveillance systems.
5. Computation of transformed and normalized NLM patterns for correlation/distance measurement can be used for high-resolution and precision identification/surveillance of specific genomic patterns of both normal and disease states.
6. Highly tunable and customizable numbers of heterogeneous genomic elements (e.g., RE, BCR/TCR/other element cluster) landscape identification/surveillance targets (type and/or locus-junction). By employing different sets of heterogeneous genomic element landscaping targets, including selection of specific restriction enzymes, the genome identification/surveillance protocol can be customizable and/or the results can be cross-checked.
7. The NLM technologies' unbiased and high-resolution landscape data characteristics provide high confidence in the identification/surveillance of specific genomes/cells.

### Repetitive Elements (RE)

Conventional genes (exome) make up about 1.2% of the human genome whereas repetitive elements (REs), both transposable and non-transposable, make up ˜75% of the human genome. REs are present in the genomes of all life forms examined so far. Different individuals within a species can share certain REs in their genomes. However, studies of the different genetic backgrounds of mice, gapes, and humans provided evidence that there are species-specific, individual-specific, tissue/cell type-specific, disease-specific, and age-dependent dynamic genomic RE landscapes with regard to their characteristics of type, copy number, and position.

### Sample Preparation

Samples for use in the methods described herein can include any of various types of biological fluids, cells and/or tissues that can be isolated and/or derived from a subject. The sample can be collected from any fluid, cell or tissue. The sample can also be one isolated and/or derived from any fluid and/or tissue that predominantly comprises blood cells.

Samples can be obtained from a subject according to any methods well known in the art. Generally, a sample that is isolated and/or derived from a subject and suitable for being assayed for genomic DNA can be used in the methods described herein. In some embodiments, the sample is, or is from, a biological fluid, e.g., blood (e.g., serum, plasma, or whole blood), semen, urine, saliva, tears, and/or cerebrospinal fluid, sweat, exosome or exosome-like microvesicles, lymph, ascites, bronchoalveolar lavage fluid, pleural effusion, seminal fluid, sputum, nipple aspirate, post-operative seroma or wound drainage fluid. In some embodiments, the sample is exosomes or exosome-like microvesicles. Methods of isolating exosomes or exosome-like microvesicles are known in the art; exemplary methods are described, e.g., in U.S. Pat. No. 8,901,284, which is incorporated by reference in its entirety. In some embodiments, the sample is isolated and/or derived from peripheral blood or cord blood. In some embodiments, the sample is from a solid tissue, e.g., a biopsy sample, from skin, tumors, or lymph nodes. Biopsy samples can include, but are not limited to, resection biopsies, punch biopsy and fine-needle aspiration biopsy (FNA).

For each sample of interest, the heterogeneous genomic element data, for example, REs, B-cell receptors (BCRs), T-cell receptors (TCRs), protocadherins, etc., with respect to each genomic element's type, copy number, and/or position, can be initially collected using various sets of probes. A series of DNA-processing protocols can be applied to the samples to obtain amplicons, for example, using polymerase chain reaction (PCR), ligation, and/or restriction digestion.

Data regarding the heterogeneous genomic elements, e.g., relating to size, sequence, and/or position, can be collected by first generating PCR amplicons from various sources. For example, a pool of amplicons can be derived from multiple PCRs, single-multiplex PCR, or PCR (single or pool of multiple reactions) following restriction digestion. A single-multiplex PCR refers to the use of PCR to amplify several different DNA sequences (e.g., multiple RE families) simultaneously (as if performing many separate PCR reactions all together in one reaction) using multiple probe sets. In some embodiments, the PCR reactions can amplify multiple regions in the genome, e.g., using primers that bind at multiple places in the genome. Typically, the PCR reactions amplify regions that include at least one heterogeneous genomic element, e.g., an RE, to produce amplicons that encompass the heterogeneous genomic element. The present methods include generating heterogeneous amplicons, i.e., a plurality of amplicons that encompass multiple heterogeneous genomic elements at different genomic positions (each amplicon includes at least one heterogeneous genomic element, and the population of amplicons includes a plurality of different amplicons, and thus includes a variety of different heterogeneous genomic elements). Thus, if the amplicons are generated using individual PCR reactions for specific, i.e., RE families, the amplicons are pooled to create a sample comprising heterogeneous amplicons.

In some embodiments, e.g., in order to produce a high-resolution identification of genomic landscapes, the heterogeneous amplicons can be digested with a set of restriction enzymes.

The heterogeneous amplicons from each genomic sample are then subjected to ddNTP termination reaction. In some embodiments, Sanger's ddNTP termination reaction is performed, and analyzed by a capillary electrophoresis sequencing instrument. Typically, the individual ddNTPs (A, T, C, G) can be labeled with fluorescent labels of different colors (emit light with different wavelengths). The ddNTP sequencing reaction is expected to produce data indicating the dideoxynucleotide termination frequency (DTF) of a specific nucleotide (A, C, G, or T) at each position that is derived from the entire population of heterogeneous amplicons.

### Dideoxynucleotide Termination Frequency Normalized Landscape Matrix (DTF-NLM)

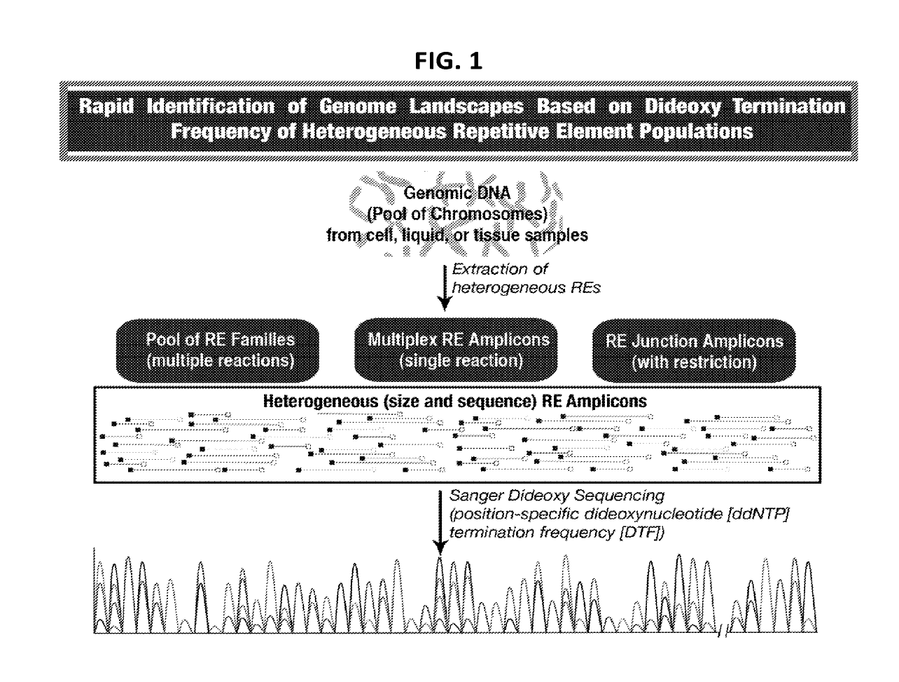

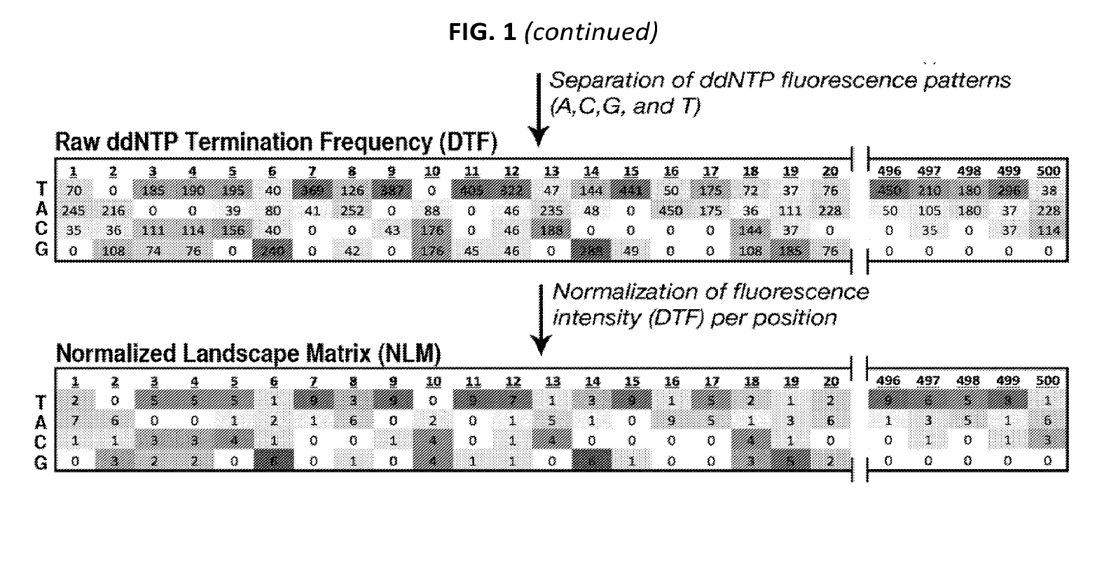

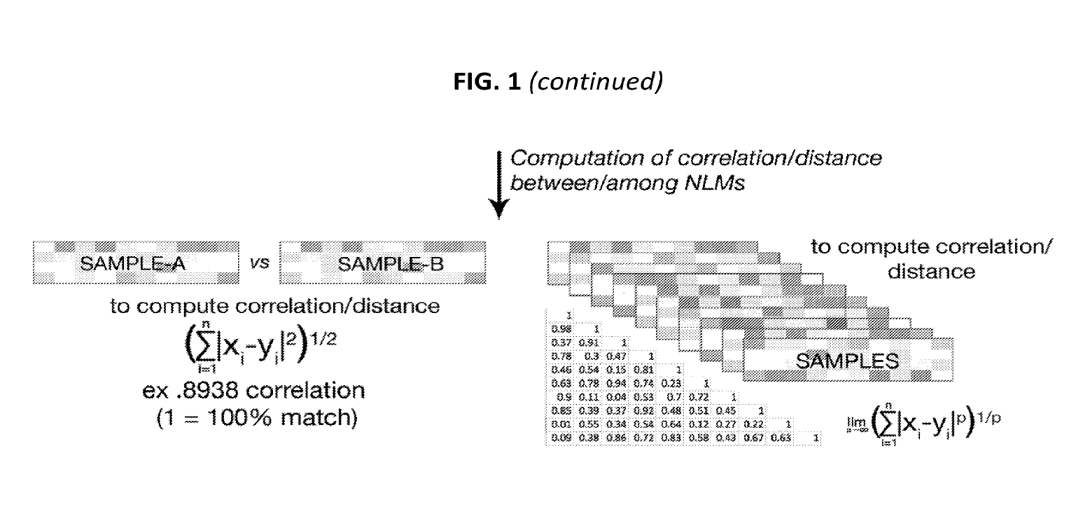

FIG. 1 illustrates one exemplary protocol of DTF sequencing and creation of a DTF normalized landscape matrix (NLM) followed by correlation/distance computation.

In conventional Sanger sequencing methods, sequencing primers that are expected to bind to only one place in the specific template DNA are used, producing a homogeneous population of amplicons. The data obtained using conventional Sanger sequencing methods therefore typically reflect one dominant fluorescence/peak at each nucleotide position in the DNA fragments produced.

Unlike in conventional Sanger sequencing methods, the present methods typically include the use of sequencing primers that bind at multiple places/targets of the population of heterogeneous genetic elements, thereby producing a heterogeneous population of DNA fragments/amplicons. Therefore, as shown in FIG. 1, during the fluorescent capillary electrophoresis sequencing, the detection device detects fluorescence intensity of dideoxynucleotides at a plurality of positions, based on binding of the sequencing primer to a plurality of different templates. Thus, at each position downstream of the primer, the present sequencing reaction generates mosaic fluorescence patterns that represent different combinations of A, C, G, and T, instead of a single nucleotide.

The intensity of fluorescence at each position is proportional to the frequency (referred to herein as the ddNTP termination frequency or DTF) of nucleotides at that position. The DTF values are transformed into a matrix of numbers (fluorescence intensities) which consist of nucleotide type (G/A/T/C) on Y-axis and position on X-axis or vice versa, as shown in FIG. 1. The intensities of fluorescence of a different number of positions are recorded. In some embodiments, the intensities of fluorescence of at least 5, 10, 50, 100, 200, 300, 400, 500, 600, or 700 positions are recorded, thus the matrix can have at least 5, 10, 50, 100, 200, 300, 400, 500, 600, or 700 columns, or at least 5, 10, 50, 100, 200, 300, 400, 500, 600, or 700 rows representing the frequency of the nucleotides at that position in the population.

The primary fluorescence intensity values can preferably be normalized by computing the relative intensity of each nucleotide at each position in order to generate a normalized landscape matrix. As used herein, normalization means adjusting values measured on different scales to a notionally common scale. In some embodiments, the relative intensity of each nucleotide at each position will be multiplied by a scaling factor, so that the sum of the relative intensity of all nucleotides at each position is a fixed number, e.g., 1, 10, 100, or any other set numbers. In some embodiments, the relative intensity of each nucleotide at each position will be multiplied by a scaling factor, so that the sum of the relative intensity of all nucleotides at all positions that are tested for each sample is a fixed number, e.g., 1, 10, 100, or any other set numbers. In some embodiments, the relative intensity of each nucleotide at each position can be adjusted by any scaling factor, as long as the sum of all elements in the NLM of a test sample is the same as the sum of all elements in a NLM of a reference sample.

### Time/Size-Intensity Landscape Matrix (TI-NLM)

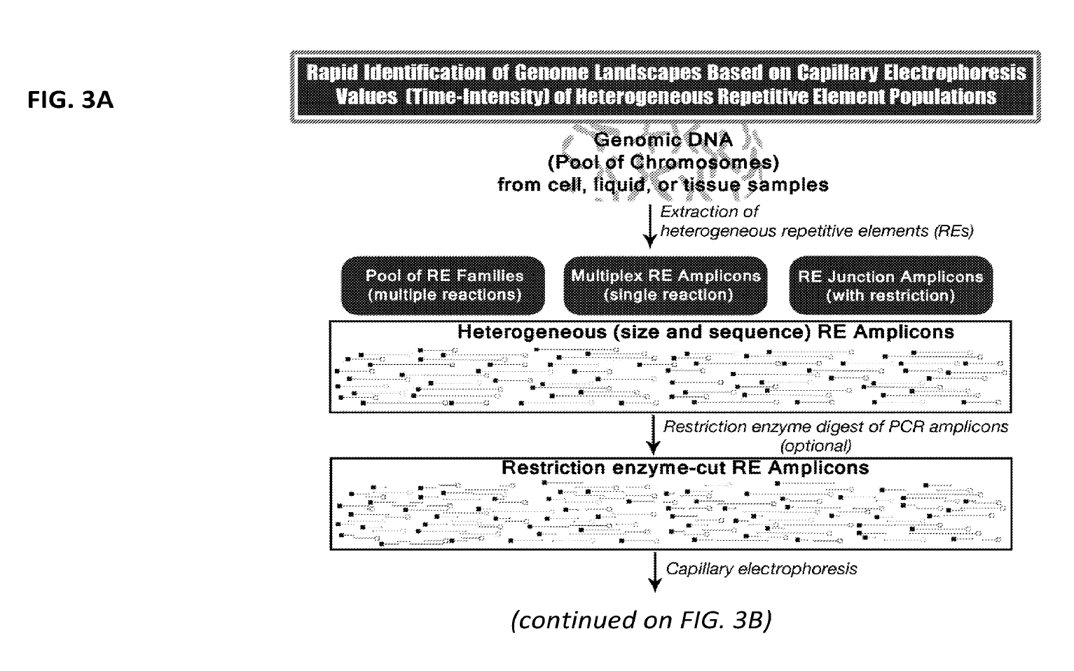

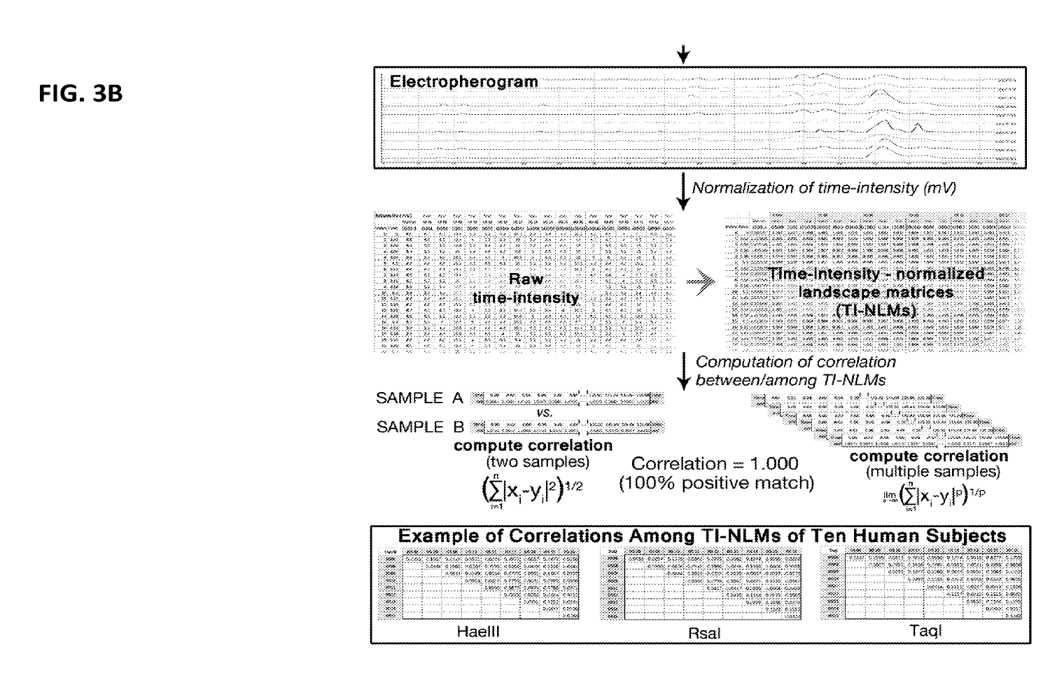

As an alternative to using DTF, Time/size-Intensity (TI) data (e.g., obtained from capillary electrophoresis) can be used. FIGS. 3A and 3B illustrate one exemplary protocol for creating and analyzing a Time/size-intensity landscape matrix, referred to herein as a TI-NLM. In these methods, a capillary electrophoresis system is used to separate the heterogeneous amplicons (optionally after a step of restriction digestion) by size through exposure to an electric field and to collect time/size-intensity data points over a specified time period. The information obtained from capillary electrophoretic analysis of each population of heterogeneous amplicons/fragments can be used to generate a graphical chart (electropherogram) or a raw numerical dataset of the amplicon/fragment intensity per time point/size. In some embodiments, the TI-NLM method uses the readouts of conventional capillary electrophoresis runs, which are time/size (second)-intensity (mV). Therefore, in some cases, there are 6000 reads of intensity (mV, (e.g., X-axis: 6000 time points (second); Y-axis: intensity (mV) value/time point). No ddNTP termination reaction is involved in the TI-NLM technology. In some embodiments, the dominant primer is labeled with a fluorescent dye which is specific for each RE family in order to fluorescently label and further amplify the landscape amplicons.

As shown in FIG. 3B, for the measurement of correlations among the heterogeneous RE populations from different genome samples, the numerical datasets of time (second)/size-intensity (mV) values obtained from the capillary electrophoresis are normalized by dividing the intensity numbers by the baseline value to create a normalized time/size-intensity landscape matrix (TI-NLM) for each sample. Using the correlation computation formulas applicable to this type of numeric matrix data, the correlation coefficients between/among the TI-NLMs, which are transformed from nucleotide sequences of heterogeneous genetic elements (e.g., RE populations), are calculated. The correlation coefficient measures the strength of the relationship between two sets of TI-NLMs which represent genomes of two individuals. A value of zero indicates no relationship. A value of 1 indicates perfect positive correlation. The correlation coefficients are then consolidated into a matrix for distance computation/phylogenetic analysis among a population of genome samples, which ultimately allows for quantitative measurement of relationship among genomes of a large and heterogeneous population of humans or other species.

Accumulation of numerically-transformed RE-landscape matrices (TI-NLMs) leads to building a machine-learnable library which can be used for precise computation of genetics correlation values, for example between two TI-NLMs, among multiple TI-NLMs, or one TI-NLM against a specific TI-NLM library (e.g., human DNA database).

### Genome Identification and Surveillance Systems

Whether produced based on DTF or TI data, the NLM pattern is specific for each genome sample, and can be used for a number of applications, including for correlation/distance computation to determine similarity/identity between two samples. In general, for correlation analysis among different genomic samples, it is important to use the same method, including the same PCR primers for the generation of heterogeneous amplicons from the original DNA sample, and the same sequencing primers for the Sanger's ddNTP sequencing reaction.

The NLM Genome Identification and Surveillance Systems can be used to rapidly and cost-effectively produce high-resolution genome identification/surveillance data by pattern computation of heterogeneous populations of genetic elements, such as REs (both transposable and non-transposable), uniquely embedded in the individual genomes.

The NLM have a number of applications. For example, the (known or unexplored) polymorphisms in species/individual-unique NLM can serve as novel identifiers of genomes from a cell or organism, with extraordinary levels of resolution and precision. The NLM can also be used as a kind of genetic fingerprint for forensic purposes. In addition, within a species, structural variations in NLM configurations can be directly applied to diagnostics as well as to the general studies of normal and disease biology.

The NLM Genome Identification and Surveillance Systems described herein can be applied to various types of heterogeneous genomic element populations. In some embodiments, the NLM Genome Identification and Surveillance Systems can be applied to RE. In some other implementations, the NLM Genome Identification and Surveillance Systems can also be applied to BCRs, TCRs, protocadherins, and other heterogeneous genomic element clusters, for example, V(D)J recombination, protocadherin rearrangement clusters.

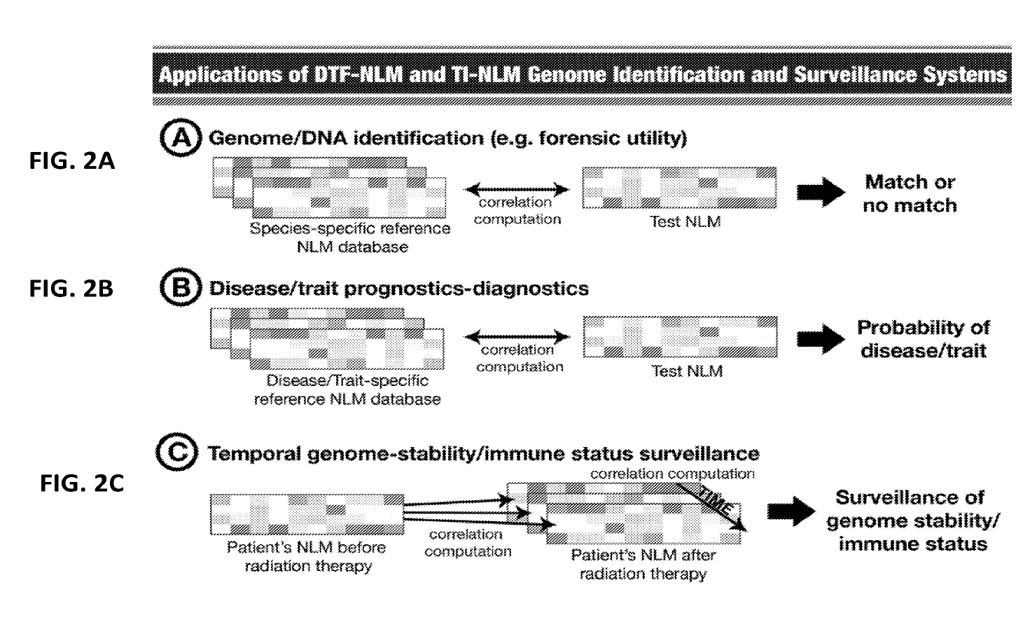

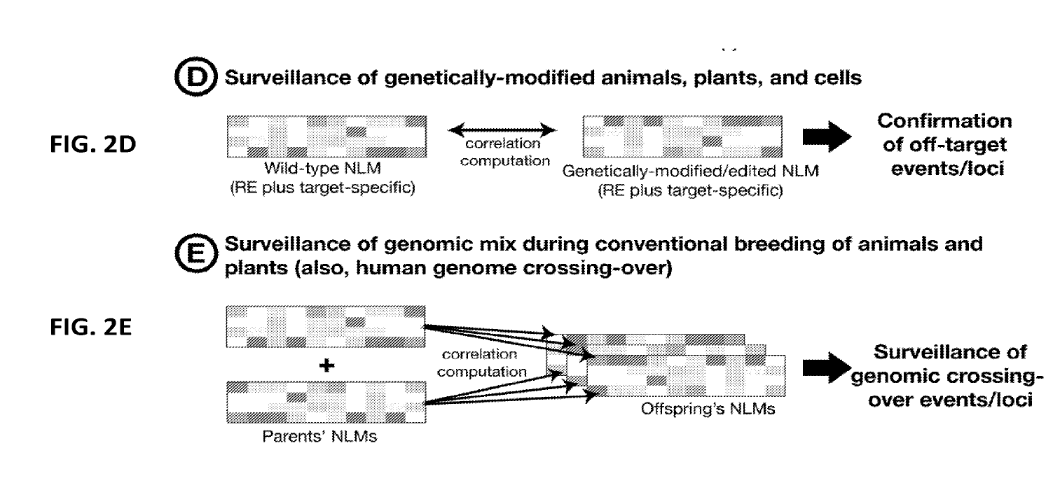

As NLM can be used to identify genomes of a cell or organism, with extraordinary levels of resolution and precision, it will further be appreciated by a person skilled in the art that the NLM Genome Identification and Surveillance Systems have various applications. These applications include:
1. Introduction of the NLM algorithms/technologies for the development of a rapid, cost-effective, highly-tunable, and precise genome identification/surveillance systems for individual humans (including monozygotic twins), animals, and plants (FIG. 2A).
2. Identification and development NLM patterns as diagnostic-prognostic markers for diseases and/or unique traits with unknown causative agents/elements (e.g., cerebral palsy, autism spectrum disorder) or without any tangible markers (e.g., ductal carcinoma in situ (DCIS) vs. breast cancer) following the establishment of disease/trait-specific NLM libraries (FIG. 2B).
3. Establishment of genome identification/surveillance/monitoring systems for laboratory animals of conventional-inbred and genetically engineered mouse strains (e.g., CRISPR-CAS9-edits, transgenics, knock-outs) based on the NLM patterns of parental strains, including wildtype controls, and offspring (FIGS. 2D-2E).
4. Establishment of a genetics identification/surveillance/monitoring systems for genetically engineered/modified/edited plants (e.g., CRISPR-CAS9-edits, transgenics, knock-outs) based on the NLM patterns of parental strains and offspring (FIGS. 2D-2E).
5. Monitoring and confirmation of the stability and compatibility of CRISPR-CAS9-edited cells (derived from humans, animals, and plants) by surveying the NLM patterns (FIG. 2D).
6. Development of diagnostics systems by identifying genomic risk factors based on the NLM patterns for a host of diseases (e.g., neonatal trisomy test, embryo screening for in vitro fertilization) with the diagnostic tools available (FIG. 2B).
7. Identification and development of prognostic genomic signatures for a range of aging-related disorders based on the NLM patterns (FIG. 2B).
8. Temporal surveillance of the genome stability and/or immune status of a patient undergoing radiation therapy or chemotherapy by examination of changes in the NLM patterns (FIG. 2C).
9. Surveillance of the effects of drugs and compounds on the genome stability and/or immune status of human patients, experimental animals, and cultured cells by examination of changes in the NLM patterns (FIG. 2C).
10. Temporal surveillance of the genome clonality/immune cell status of tumor lesions of patients (e.g. leukemia) undergoing treatment by examining changes in the NLM patterns (FIG. 2C).
11. Establishment of species/strain/individual-specific as well as disease-specific NLM databases, which can be used to organize, and utilize the constantly expandable RE/BCR/TCR/other genomic cluster landscape data (FIG. 2A).

### Computer Implementation

The NLM can be stored, e.g., in electronic media such as a flash drive as well as on paper or other media. The NLM can also be represented electronically on a monitor or screen, such as on a computer monitor, a mobile telephone screen, or on a personal digital assistant (PDA) screen. The NLM can also be analyzed and compared by computer in digital, electrical form without the need for a tangible printout or image represented on a computer or other screen or monitor.

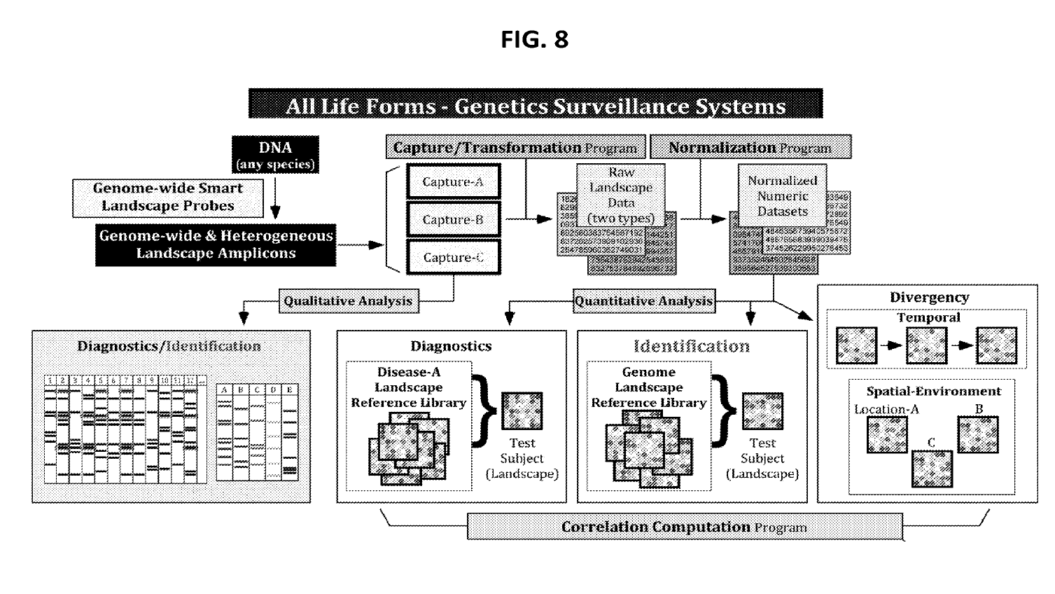

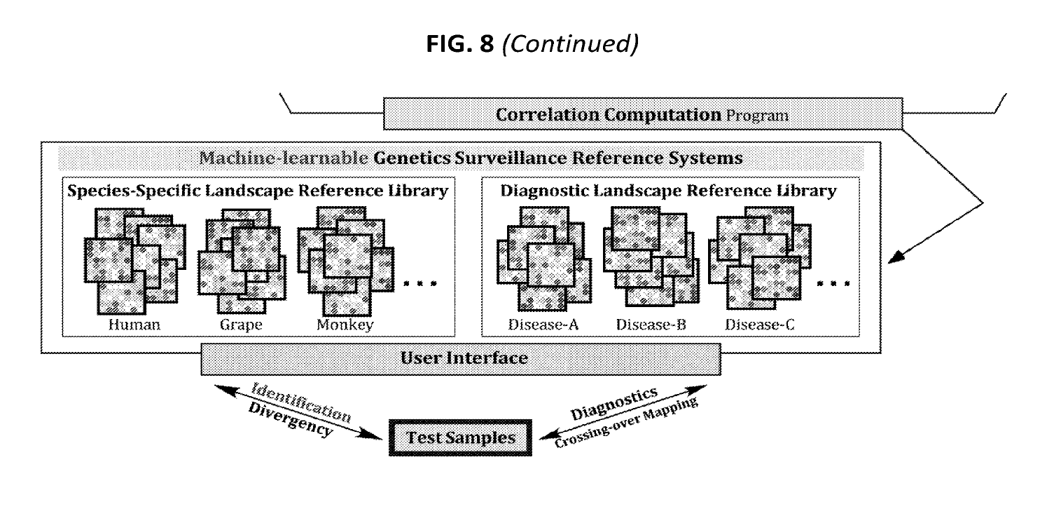

The NLM can be generated using a computer system, e.g., as described in WO 2011/146263 and FIG. 8 therein, which is a schematic diagram of one possible implementation of a computer system 1000 that can be used for the operations described in association with any of the computer-implemented methods described herein. The system 1000 includes a processor 1010, a memory 1020, a storage device 1030, and an input/output device 1040. Each of the components 1010, 1020, 1030, and 1040 are interconnected using a system bus 1050. The processor 1010 is capable of processing instructions for execution within the system 1000. In some embodiments, the processor 1010 is a single-threaded processor. In another implementation, the processor 1010 is a multi-threaded processor. The processor 1010 is capable of processing instructions stored in the memory 1020 or on the storage device 1030 to display graphical information for a user interface on the input/output device 1040.

The memory 1020 stores information within the system 1000. In some embodiments, the memory 1020 is a computer-readable medium. The memory 1020 can include volatile memory and/or non-volatile memory.

The storage device 1030 is capable of providing mass storage for the system 1000. In some embodiments, the storage device 1030 is a computer-readable medium. In various different implementations, the storage device 1030 may be a disk device, e.g., a hard disk device or an optical disk device, or a tape device.

The input/output device 1040 provides input/output operations for the system 1000. In some embodiments, the input/output device 1040 includes a keyboard and/or pointing device. In some embodiments, the input/output device 1040 includes a display device for displaying graphical user interfaces.

The methods described can be implemented in digital electronic circuitry, or in computer hardware, software, firmware, or in combinations of them. The methods can be implemented in a computer program product tangibly embodied in an information carrier, e.g., in a machine-readable storage device, for execution by a programmable processor; and features can be performed by a programmable processor executing a program of instructions to perform functions of the described implementations by operating on input data and generating output. The described methods can be implemented in one or more computer programs that are executable on a programmable system including at least one programmable processor coupled to receive data and instructions from, and to transmit data and instructions to, a data storage system, at least one input device, and at least one output device. A computer program includes a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. Computers include a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, computers and networks that form the Internet.

The computer system can include clients and servers. A client and server are generally remote from each other and typically interact through a network, such as the described one. The relationship of client and server arises by virtue of computer programs running on the respective computers and having a client-server relationship to each other.

The processor 1010 carries out instructions related to a computer program. The processor 1010 may include hardware such as logic gates, adders, multipliers and counters. The processor 1010 may further include a separate arithmetic logic unit (ALU) that performs arithmetic and logical operations.

### Distance and Correlation

For the identification and/or surveillance, the NLM from individual genome samples are subjected to correlation/distance computation using established mathematical formulas: between two NLMs, among multiple NLMs, or one NLM against a specific NLM library. These mathematical operations can be performed in a computer system 1000 as described in this disclosure.

In some embodiments, the distance (d) between two DTF-NLMs can be calculated based by the following equation:

$$d = {( {\sum }_{i = 1}^{n} {( {X}_{i} - {Y}_{i} )}^{2} )}^{1 / 2}$$

In this equation, n is the total number of elements in the NLM. The letter i indicates the ith element in the NLM. Thus the value of i ranges from 1 to n. Furthermore, Xᵢ is the value of the ith element in the NLM obtained from a test genome sample. Yᵢ is the value of the ith element in the NLM from a reference genome sample.

In some embodiments, the distance (d) among multiple DTF-NLMs can be calculated by the following equation:

$$d = {lim}_{P \to ∞} {( {\sum }_{i = 1}^{n} {\vert {X}_{i} - {Y}_{i} \vert}^{P} )}^{1 / P}$$

In some embodiments, the correlation (r) among multiple TI-NLMs can be calculated by the following equation:

$${r}_{xy} = \frac{{\sum }_{i = 1}^{n} ( {x}_{i} - \overline{x} ) ( {y}_{i} - \overline{y} )}{{ns}_{x} {s}_{y}} = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2} {\sum }_{i = 1}^{n} {( {y}_{i} - \overline{y} )}^{2}}}$$

where x̅ and y̅ are the sample means of X and Y, and Sₓ and S_{y} are the sample standard deviations of X and Y. Xᵢ is the value of the ith element in the NLM obtained from a test genome sample. Yᵢ is the value of the ith element in the NLM from a reference genome sample.

The correlation/distance values, which are derived from these pattern computations, can be directly applied for the identification and/or surveillance of test genome samples. In some embodiments, a NLM can be generated for a subject who is undergoing treatment for a disease, e.g., cancer, e.g., before and after the treatment, and the distance can be calculated between the two. A large distance would indicate that the treatment is destabilizing the DNA. In some embodiments, a combinatorial interpretation of the NLM data obtained from two or more RE families, probes, or restriction enzymes can be implemented for a final confirmation of the critical data sets (e.g., forensic DNA identification).

In some embodiments, accumulation of species-specific NLM data will increase the accuracy for the identification and surveillance of genome samples of all life forms.

### Reference Threshold

In the present methods, the NLM technologies compute the distance/correlation directly between/among samples; a reference threshold (i.e., a preselected level of distance or correlation) can be used to determine whether two samples are correlated or close enough to be deemed identical or have the same characteristics. For example, when the distance between the NLM of a test subject and the NLM of a reference subject is less than a reference threshold distance, it can be determined that the two subjects have the same characteristics. For example, in some embodiments, when the distance between the NLM of a test subject and the NLM of a reference subject is less than a reference threshold distance, it can be determined that the two subjects have the same genetic identify. In some embodiments, when the distance between the NLM of a test subject and the NLM of a reference subject having a particular trait (e.g., a disease, a genetic risk factor) is less than a reference threshold distance, it can be determined that the test subject is likely to have the same trait (e.g., a disease, a genetic risk factor). When the correlation between the NLM of a test subject and the NLM of a reference subject is higher than a reference threshold distance (e.g., 0.6, 0.7, 0.8, or 0.9), it can be determined that the two subjects have the same characteristics. For example, in some embodiments, when the correlation between the NLM of a test subject and the NLM of a reference subject is higher than a reference threshold correlation, it can be determined that the two subjects have the same genetic identify. In some embodiments, when the correlation between the NLM of a test subject and the NLM of a reference subject having a particular trait (e.g., a disease, a genetic risk factor) is higher than a reference threshold correlation, it can be determined that the test subject is likely to have the same trait (e.g., a disease, a genetic risk factor).

The reference threshold distance or correlation used in the present methods can be determined empirically or by any other means known in the art. In some embodiments, the reference threshold distance or correlation is determined by testing a large number of subjects, wherein the reference threshold distance or correlation is selected for highest accuracy, highest positive predictive value, or highest negative predictive value.

The threshold distance or correlation can be similarly applied to NLM derived from all kinds of samples, including e.g., samples from bacteria, cells, tissues, organs, or all kinds of organisms. For example, if the distance between the NLM of a test cell and the NLM of a reference cell is less than a reference threshold distance (or the correlation between the NLM of a test cell and the NLM of a reference cell is higher than a reference correlation), it can be determined that the test cell and the reference cell are likely to have the same genetic identity (e.g., belonging to the same cell line). If the distance between the NLM of a test bacterium and the NLM of a reference bacterium is less than a reference threshold distance (or the correlation between the NLM of a test bacterium and the NLM of a reference bacterium is higher than a reference correlation), it can be determined that the test bacterium and the reference bacterium are likely to have the same genetic identity (e.g., belonging to the same species). In some other cases, when the distance between the NLM of a test sample (e.g., cultured cells) and the NLM of a reference sample is greater than a reference threshold distance (or the correlation between the NLM of the test sample and the NLM of a reference sample is less than a reference correlation), it can be determined that the test sample is likely to have contamination (e.g., by bacteria, by other types of cells).

### EXAMPLES

The invention is further described in the following examples, which do not limit the scope of the invention described in the claims.

### Example 1: Time/Size-Intensity Landscape Matrix

Each human has a unique genomic landscape formed by the inherent diversity and/or acquired activity of repetitive elements (REs), including human endogenous retroviruses (HERVs), within their genome. This genomic RE landscape can function as a unique identifier of the individual's genome and phenotype. Experiments were performed to create time/size-intensity landscape matrices for 9 human subjects.

Heterogeneous RE samples were obtained using a collection of primer sets by polymerase chain reaction (PCR). In this example study, the following primers were used:

Forward:
(SEQ ID NO: 1)
AGG CAA GAG ACT GAA GGC AC
Reverse:
(SEQ ID NO: 2)
GTA GGG CTG GAC CCT ACA.

In order to produce a high-resolution identification of genomic landscapes, the heterogeneous RE amplicons were then digested by restriction enzymes respectively: RsaI, TaqI, and HaeIII.

The capillary electrophoresis system separated the PCR amplicons/restriction fragments by size through exposure to an electric field and collected time/size-intensity data points from the detection of the first signal to about 135 second after.

The information obtained from capillary electrophoretic analysis of each population of heterogeneous RE amplicons/fragments were used to generate a graphical chart (electropherogram) or a raw numerical dataset of the amplicon/fragment intensity per time point/size (FIGS. 3A-3B). One particular dataset includes the intensity of a marker for each subject at 0.02 second interval for a period of 135.08 seconds.

For the measurement of correlations among the heterogeneous RE populations from different genome samples, the numerical datasets of time (second)/size-intensity (mV) values were normalized by dividing the intensity numbers by the baseline value to create a normalized time/size-intensity landscape matrix (TI-NLM) for each sample.

Using the correlation computation formulas, the correlation coefficients between/among the TI-NLMs, which were transformed from nucleotide sequences of heterogeneous RE populations, were calculated (FIGS. 3A-3B). A value of zero indicates no relationship and a value of 1 indicates perfect positive correlation. These results are shown in Tables 1-3. The correlation coefficient measures the relationship between two sets of TI-NLMs which represent genomes of two individuals. For example, in Table 1, HS06 and HS15 has a high correlation. Similar results are observed for HS06 and HS15 in Tables 2 and 3.

TABLE 1
Correlation matrices for 9 human genome samples*

| RsaI | HS 08 | HS 09 | HS 10 | HS 11 | HS 12 | HS 13 | HS 14 | HS 15 | HS 16 |
|---|---|---|---|---|---|---|---|---|---|
| HS06 | 0.0433 | −0.0054 | 0.1378 | −0.0340 | 0.0378 | 0.3062 | 0.1348 | 0.9190 | 0.0338 |
| HS08 | | −0.0360 | 0.0626 | −0.0542 | 0.6990 | 0.0436 | 0.1398 | 0.0404 | 0.9561 |
| HS09 | | | −0.0044 | 0.6010 | −0.0255 | −0.0139 | −0.0007 | −0.0027 | −0.0276 |
| HS10 | | | | −0.0346 | 0.0796 | 0.0847 | 0.8875 | 0.1320 | 0.0547 |
| HS11 | | | | | −0.0417 | −0.0317 | −0.0505 | −0.0304 | −0.0428 |
| HS12 | | | | | | 0.0430 | 0.1118 | 0.0375 | 0.8065 |
| HS13 | | | | | | | 0.0860 | 0.1988 | 0.0376 |
| HS14 | | | | | | | | 0.1262 | 0.1137 |
| HS15 | | | | | | | | | 0.0328 |

*RE amplicons were treated with restriction enzymes RsaI.

TABLE 2
Correlation matrices for 9 human genome samples*

| TaqI | HS 08 | HS 09 | HS 10 | HS 11 | HS 12 | HS 13 | HS 14 | HS 15 | HS 16 |
|---|---|---|---|---|---|---|---|---|---|
| HS06 | 0.1310 | 0.1586 | 0.0511 | 0.9852 | 0.0950 | 0.1354 | 0.0418 | 0.8279 | 0.1755 |
| HS08 | | 0.1005 | 0.0657 | 0.1414 | 0.5986 | 0.0865 | 0.0531 | 0.2298 | 0.9634 |
| HS09 | | | 0.0291 | 0.1267 | 0.1060 | 0.9693 | 0.0207 | 0.1043 | 0.1152 |
| HS10 | | | | 0.0497 | 0.1255 | 0.0362 | 0.6808 | 0.0548 | 0.0655 |
| HS11 | | | | | 0.0911 | 0.1111 | 0.0397 | 0.8947 | 0.1914 |
| HS12 | | | | | | 0.1157 | 0.0615 | 0.1315 | 0.6095 |
| HS13 | | | | | | | 0.0187 | 0.1148 | 0.1195 |
| HS14 | | | | | | | | 0.0366 | 0.0397 |
| HS15 | | | | | | | | | 0.3282 |

*RE amplicons were treated with restriction enzymes TaqI.

TABLE 3
Correlation matrices for 9 human genome samples*

| HaeIII | HS 08 | HS 09 | HS 10 | HS 11 | HS 12 | HS 13 | HS 14 | HS 15 | HS 16 |
|---|---|---|---|---|---|---|---|---|---|
| HS06 | 0.0251 | 0.1907 | 0.0919 | 0.5571 | 0.0231 | 0.4977 | 0.0857 | 0.9877 | 0.0268 |
| HS08 | | 0.0368 | 0.0568 | 0.0280 | 0.7078 | 0.0294 | 0.0409 | 0.0226 | 0.8941 |
| HS09 | | | 0.0833 | 0.6349 | 0.0334 | 0.6777 | 0.0992 | 0.1607 | 0.0397 |
| HS10 | | | | 0.0903 | 0.0353 | 0.0760 | 0.9091 | 0.0960 | 0.0860 |
| HS11 | | | | | 0.0260 | 0.9879 | 0.0874 | 0.4788 | 0.0301 |
| HS12 | | | | | | 0.0275 | 0.0282 | 0.0224 | 0.4751 |
| HS13 | | | | | | | 0.0731 | 0.4251 | 0.0331 |
| HS14 | | | | | | | | 0.0893 | 0.0536 |
| HS15 | | | | | | | | | 0.0260 |

*RE amplicons were treated with restriction enzymes HaeIII.

### Example 2: Rapid, Precise, Cost-Effective, and Machine-Learnable Identification/Surveillance of Microbes (RaPIdMicro)

A microbial identification-surveillance system is tested on E. coli as an example. The system is highlighted by: 1) rapid and high-resolution collection of a population of genomic landscape amplicons using a single or multiple repetitive elements (RE) probes, 2) transformation of the population of heterogeneous RE amplicons into a numeric matrix followed by normalization, and 3) correlation computation of the normalized RE landscape matrices between/among genomes of interest in order to produce quantifiable, precise, and machine learnable genetic identification-surveillance values.

Establishment of a Library of REs from Reference E. coli Genomes

Genomic RE landscapes (RE type and genomic position) are expected to be highly heterogeneous among the microbial population due to REs' inherent diversity and acquired activity. The in silico RE mining study is designed to establish an RE library by systematically cataloging RE landscape data from E. coli genomes. Public RE databases and literature can be surveyed to retrieve reported REs followed by size and type grouping. REs in each size or type group are aligned to define conserved regions in order to design probes for RE mining from NCBI's E. coli genome databases using the Basic Local Alignment Search Tool (BLAST). In addition to this mining strategy using the RE probes and BLAST, an RE mining program (REMiner) which identifies and maps REs de novo in a genome sequence primarily based on the seeding and penalty settings in conjunction with the REViewer visualization program can be used. REMiner and REViewer are described, e.g., in Chung, Byung-Ik, et al. “REMiner: a tool for unbiased mining and analysis of repetitive elements and their arrangement structures of large chromosomes.” Genomics 98.5 (2011): 381-389; and You, Ri-Na, et al. “REViewer: A tool for linear visualization of repetitive elements within a sequence query.” Genomics 102.4 (2013): 209-214, each of which is incorporated by reference in its entirety.

Each RE Locus from the BLAST and REMiner Surveys can be Examined to collect the sequence and genomic position information as well as annotations for neighboring genes. The REs collected can be classified into families by multiple alignment and clustering analyses followed by organization into the RE library of E. coli.

### Designing Probes Capable of Amplifying a Large Population of Heterogeneous REs

For each RE family in the RE library of E. coli, probing regions are defined and corresponding RE landscape primer sets are designed. A detailed description of repetitive elements in prokaryotic genomes (e.g., genomes of E. coli) is described, e.g., in Lupski, James R., and GEORGE M. Weinstock. “Short, interspersed repetitive DNA sequences in prokaryotic genomes.” Journal of bacteriology 174.14 (1992): 4525, which is incorporated by reference herein in its entirety. Some positions in these primers contain degeneracy in order to maximize the coverage of REs with similar sequences. Two types of probing regions are considered when the landscaping primer sets are designed: (1) hyper-variable regions within each RE family for computing REs' inherent polymorphism (type) using standard PCR and (2) conserved regions for computing the REs' inherent polymorphism (type and position) and acquired activity (type and position) using inverse-PCR (I-PCR).

E. coli and Other Microbial Samples Subjected to Genome Landscaping Analyses

Ten biosafety level-1 E. coli strains, including the DH5a strain, as well as four biosafety level-1 bacterial types (Streptococcus, Pseudomonas, Staphylococcus, and Bacillus) are tested by the RaPIdMicro system and are placed into one or all of the following landscaping study groups.

A. Optimization of Microbial Landscape Detection and Resolution:

A series of E. coli (DH5a) cultures with different concentrations are added into human whole blood (HWB) from a blood bank, which represents a microbial host environment, in order to test protocols relevant to collecting RE landscape amplicons, including size spectrum of amplicons, determination of detection sensitivity, and resolution of the prototype RaPIdMicro system.

B. Construction of a RE Landscape Reference of E. coli:

Ten E. coli strains are added into HWB individually to prepare cells for creating a prototype RE landscape reference of E. coli for identification-surveillance of microbial species and/or strains.

C. Identification of E. coli in a Mixed Microbial Population:

To evaluate the specificity of the RaPIdMicro system at the species level, HWB are added with the four bacterial types listed above ((Streptococcus, Pseudomonas, Staphylococcus, and Bacillus)) plus E. coli-DH5a. E. coli-DH5a is the identification target using the RE landscape reference of E. coli while the RE landscape matrices from non-Escherichia samples serve as negative correlation controls.

Genomic DNAs are isolated from the HWB samples added with E. coli and/or other bacteria, concentrations are measured, and their quality is evaluated by confirming the high molecular weight banding pattern prior to normalization to 20 ng/μl. The isolated genomic DNA samples is subjected to the RE landscape analyses.

Collection of a Population of RE Landscape Amplicons and Transformation into a Numeric Matrix

Each microbial species/strain has a dynamic and unique set of genomic RE landscapes which are formed by the inherent diversity and acquired activity of REs. These dynamic and heterogeneous RE landscapes function as novel identifiers of each microbe's innate and dynamic genomes. The following RE landscaping and computation protocols are applied to the individual microbial cultures.

A. Collection of a Population of RE Amplicons:

A population of heterogeneous REs (type and position), embedded in the microbial genomes, are obtained using landscaping primer sets which are designed to amplify specific RE families (standard PCR) and their insertion junctions (I-PCR). DNA-processing protocols, such as restriction digestion and ligation, are employed before I-PCR amplification. The heterogeneous (size and sequence) RE landscape amplicons from each culture can be typically collected as: 1) RE landscape amplicons derived from multiple PCRs with standard primers, 2) RE landscape amplicons from single-multiplex PCR with standard primers, and 3) RE junction-landscape PCR amplicons (single or pool of multiple reactions) using I-PCR primers. A set of PCR parameters are evaluated in order to render optimal resolution and size-spectrum of RE landscape amplicons.

B. Numeric Transformation of RE Landscape Amplicons by Dideoxynucleotide (ddNTP)-Termination:

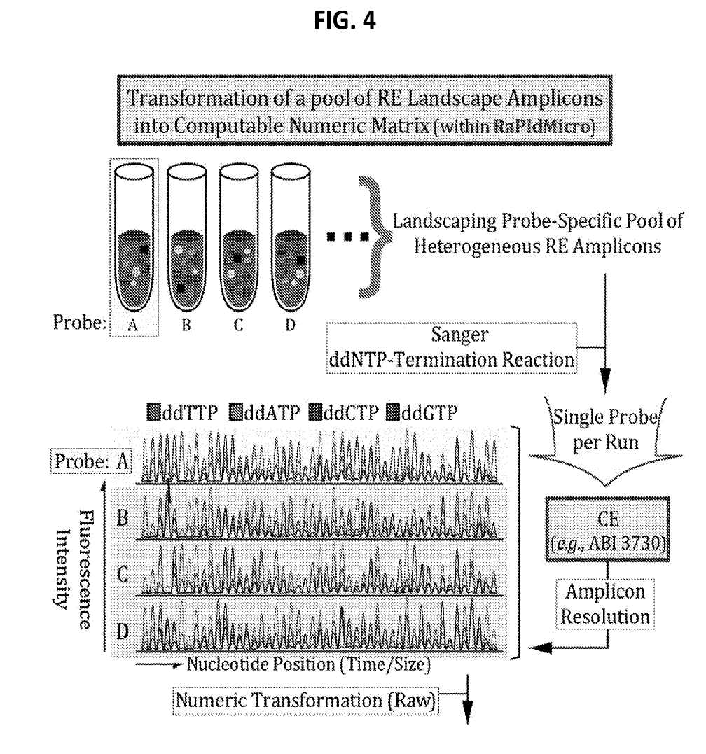

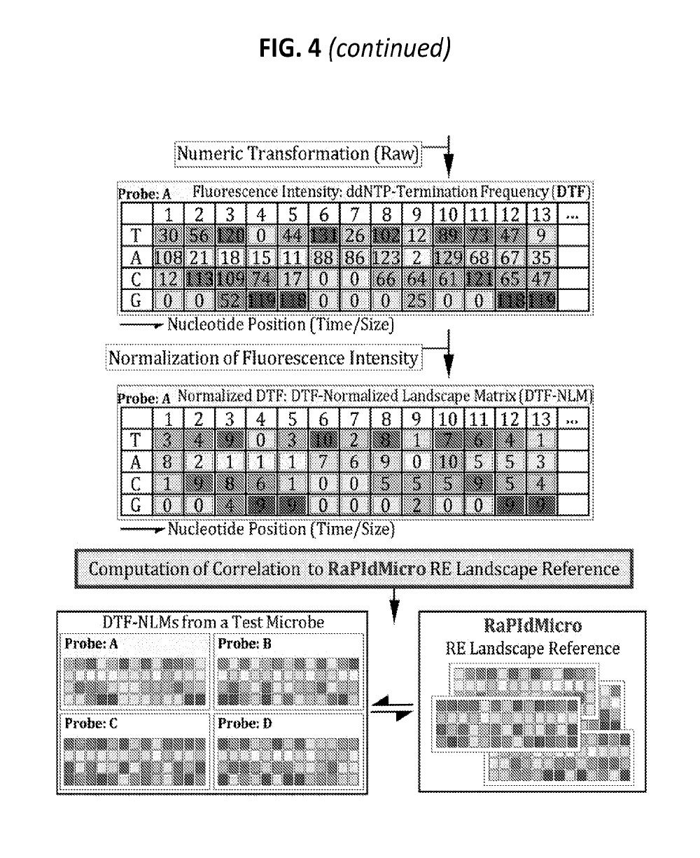

The RE landscape amplicons are then subjected to a Sanger's ddNTP-termination reaction followed by resolution of the nucleotide position-specific occurrence frequency of ddNTP-termination of individual nucleotides using four-color-fluorescent capillary electrophoresis (CE) equipment (e.g., ABI 3730 DNA Analyser, Applied BioSystems, Foster City, Calif.) (FIG. 4). Each ddNTP type is labeled with a fluorescein of a unique wavelength. The ddNTP-termination reactions generate data with regard to the ddNTP-termination frequency (DTF) of individual nucleotides (A, C, G, or T) per nucleotide position, which is counted from the priming site and thus, shared by the entire population of heterogeneous RE molecules. In contrast to conventional Sanger sequencing data, which typically depicts one dominant fluorescent peak at each nucleotide position, the DTF resolution of a heterogeneous RE population generates a mosaic of peaks that represents the combination of A, C, G, and T at each position. The fluorescence intensity is directly converted to the DTF of the respective nucleotides at each position. The compiled D a values of a heterogeneous RE population, which are recorded as intensity of fluorescence with different wavelengths, are transformed into a matrix of numbers (fluorescence intensities) which consist of an X-Y plot of nucleotide position (variable number) and type (four nucleotides).

### Normalization and Correlation Computation of Numeric RE Landscape Matrices

To prepare the numeric RE landscape matrices (DTFs) for correlation computation, the DTFs' primary fluorescence intensity values are normalized by calculating the relative intensity of each nucleotide at each position (FIG. 4). A DTF's normalized landscape matrix (DTF-NLM) that is unique for each microbial culture is now ready for the downstream correlation computation. For microbial identification and surveillance, the DTF-NLMs from individual cultures are subjected to correlation computation using a collection of established mathematical formulas: between two DTF-NLMs (confirmation), among multiple DTF-NLMs (temporal and spatial divergence), or one DTF-NLM against a specific DTF-NLM-landscape reference (identification and surveillance). The correlation coefficient measures the strength of the relationship between two DTF-NLMs, which represent two microbial genome/culture samples. A value of zero indicates no relationship. A value of 1 indicates perfect positive correlation. Furthermore, for the quantitative measurement of relationships among the genomes of a heterogeneous population of microbes, the correlation coefficients of individual pairs are consolidated into a matrix for distance computation followed by clustering/classification.

### Construction of a Prototype RaPIdMicro System, Including RE Landscape Reference of E. Coli

The DTF-NLMs of the 10 E. coli strains are organized into a RE landscape reference of E. coli within a prototype RaPIdMicro DBMS which can compute the correlation of a query RE landscape matrix (DTF-NLM) derived from a test microbe, against the reference. Accumulation of RE landscape matrices for a range of microbes at genus, species, and/or strain levels leads to establishing machine learnable RAPIDmicro systems for the entire microbial world and/or individual genus/species for rapid, precise, and cost-effective computational identification and surveillance of microbes.

### Expected Results and Alternative Approach

The primary outcome is the development of a suite of reagents (RE landscaping probes), protocols, algorithms, RE landscape reference of E. coli, and a DBMS, which are the core components of the prototype RaPIdMicro system. In addition, performance of the RaPIdMicro system is initially evaluated by testing its ability to differentially identify E. coli from the other four bacterial types. More than one RE landscape primer set can be employed for cross-confirmation within the RaPIdMicro system (FIG. 4). Furthermore, the RE landscape-based RaPIdMicro system can significantly improve the confidence level of identification. For instance, implementing 32 RE loci information derived from a landscaping reaction using a single primer set, instead of the data from 16 short tandem repeat loci (current standard for human identification with 16 primer sets), can decrease the likelihood of misidentification by a factor of one billion (1×10⁹), using the assumption of independence and the multiplication rule. The probability of false positives can also decrease based on conditional probability when combined with other lines of information derived from independent primer sets. Together, the resources produced in this project can be the foundation for developing a range of machine learnable RaPIdMicro systems which focus on either single or multiple microbial species. Furthermore, the RaPIdMicro system can be applied to a range of fields, such as medicine, food and agriculture, and environment as well as for identification and surveillance of the humans, animals, and plants.

As an alternative to the ddNTP-termination strategy of numeric transformation of RE landscape amplicons, the RE amplicons can be subjected to asymmetric PCR with the dominant primer labeled with a fluorescent dye which is specific for each RE family in order to fluorescently label and further amplify the landscape amplicons. Subsequently, the size and intensity profiles of the population of heterogeneous RE landscape amplicons are resolved by conventional CE which yields thousands of time (e.g., every 0.2 seconds)/size-intensity data points over a typical run period. The time/size-intensity datasets, which are transformed from the heterogeneous population of RE landscape amplicons, are ready for normalization followed by correlation computation.

### Example 3: Evaluate the Sensitivity and Specificity of the RaPIdMicro Tool by Correlating a Specific Microbe's RE Landscape to the RE Landscape Reference Library

In this study, the RaPIdMicro system is evaluated with regard to its ability to differentially identify individual strains of a microbial species using a range of E. coli strains that are added into HWB. The RE landscape matrices (DTF-NLMs) of 10 E. coli strains collected from various culture passages are generated using the RaPIdMicro RE landscaping probes, protocols, and algorithms as described in Example 2, and are further subjected to correlation computation using the RE landscape reference of E. coli to obtain differential identification values.

Study Design for Differential Identification of E. coli Strains

The same 10 E. coli strains, which are used in Example 2, are subjected to the following treatment before they are collected for genomic DNA isolation. For each of the 10 E. coli strains, cultures from five different passages (1, 5, 10, 20, and 40) are added into HWB individually. Quintuplet samples of each E. coli stain are used to evaluate whether the RaPIdMicro system is able to discern different E. coli strains with precision and reproducibility by correlation computation against the system's RE landscape reference of E. coli. Moreover, temporal (passage number-dependent) variations in E. coli genomic landscapes can be quantified. Genomic DNAs are collected from each HWB-E. coli strain sample for RE landscape analyses.

### Generation of Normalized RE Landscape Matrices (DTF-NLMs) Followed by Strain Identification

Using the same RE landscaping probes, protocols, and algorithms which are applied to construct the RE landscape reference of E. coli: (1) heterogeneous landscape amplicons are collected from E. coli genomes followed by transformation into numeric matrices of ddNTP-termination frequency (DTF), (2) the raw numeric matrices are normalized (DTF-NLM) to prepare them for correlation analysis by calculating the relative intensity of each nucleotide at each position, and (3) the DTF-NLMs from individual E. coli strains are subjected to correlation computation against the RE landscape reference of E. coli in the prototype RaPIdMicro system, in order to differentially identify the E. coli strains. In addition, the passage number-dependent variations in RE landscapes of individual E. coli strains are measured.

### Expected Results and Alternate Approach

To evaluate the accuracy and resolution of the RE landscape correlation values, a series of computation simulation studies are performed using in silico-generated raw numeric RE landscapes and/or DTF-NLMs. In addition, analytical protocols, which involve combinatorial interpretation of the DTF-NLM datasets obtained from two or more RE landscaping probes, are implemented in order to confirm identification and surveillance values.

RE landscapes are expected to be different depending upon microbial species and strains, and culture passages/conditions. It is expected that the prototype RaPIdMicro system produces correlation values which are specific enough to differentially identify the 10 E. coli strains. In addition, the landscape correlation values can be sensitive enough to detect temporal variations in RE landscapes depending on the culture schedule. The machine learnable RaPIdMicro system is expected to perform 1) rapid, precise, and cost-effective surveillance of genetic identity of pathogenic microbial species, strains, and variants (temporal and spatial) and 2) high-resolution surveillance of genetic drifts in bacteria.

### Example 4: Determining Human and Mouse Cell Lines with Regard to Identity, Divergence (Temporal and Spatial), and Contamination

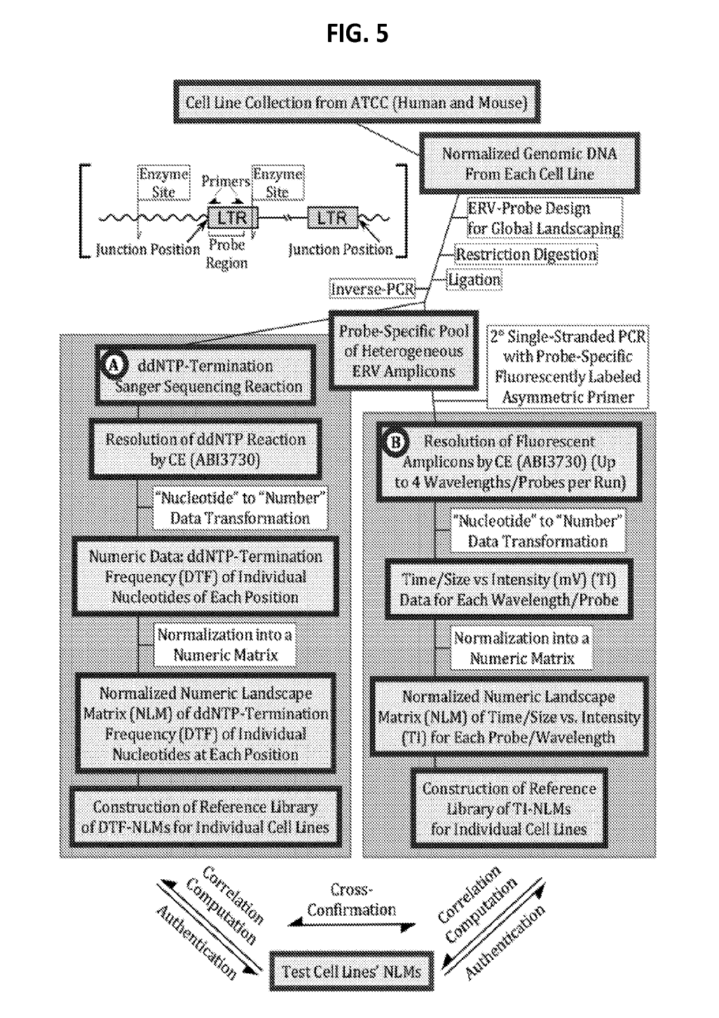

A genome surveillance protocols and algorithms (“GST”) is developed. The system is highlighted by (FIG. 5) for 1) rapid and cost-effective collection of a large population of heterogeneous TRE-landscape amplicons/fragments using proprietary probes, 2) transformation of a heterogeneous population of TRE-landscape molecules into a matrix of numbers using proprietary algorithms, 3) normalization of the raw numbers in a matrix, and 4) correlation computation of the normalized numeric TRE-landscape matrices between/among genomes of interest in order to produce quantifiable and machine-learnable genetics surveillance-identification values.

### Refinement of HERV and MuERV Libraries

It is expected that the genomic HERV/MuERV landscapes among different humans and mouse strains are immensely heterogeneous primarily due to their high-levels of inherent diversity. HERV and MuERV libraries are built by surveying the NCBI's reference genomes (human-build-37; mouse-Build 36). It is important to have access to comprehensive HERV/MuERV libraries for designing efficient landscaping probe sets. In this example, the most recent versions of the human and mouse genome databases in silica are surveyed to mine new HERVs and MuERVs, including their position information, using BLAST probes designed from current libraries in order to update the HERV and MuERV libraries.

Currently, the NCBI's reference human and mouse genomes are determined to be the best-assembled with regard to both quality and quantity; therefore, the NCBI reference genomes can serve as the primary resource for this mining, in addition to other well-assembled genomes. Although the identity threshold can vary during the HERV-MuERV mining using the NCBI's BLAST program and/or similar genome mining tools, it can be initially set to 80%. The BLAST hits from the genome-wide HERV-MuERV surveys are examined to collect the following information: structure, sequence (full or partial), and position of individual HERVs/MuERVs. The newly identified HERV/MuERV datasets are updated into the HERV and MuERV libraries. The updated HERV and MuERV libraries are interrogated to design systematic and comprehensive probes for landscaping the genomes of cell lines.

### Designing of Probes (at Least 100) Capable of Amplifying Heterogeneous Populations of HERVs/MuERVs

The HERVs and MuERVs in the updated libraries are categorized into subfamilies by multiple alignment and clustering analyses. Within the individual HERV/MuERV families, at least 100 probe regions and corresponding primer sets are designed primarily from the long terminal repeat (LTR) sequences for each species. Some positions within these primers contain degeneracy in order to maximize the coverage of HERVs and MuERVs. Two types of probe regions are considered when the HERV/MuERV primer sets are designed: 1) hyper-variable LTR regions for standard PCR and 2) inverse-PCR (I-PCR) probes on LTRs.

### Selection and Processing of Cell Lines for Genome Landscaping Analyses: Identity, Divergence (Temporal and Spatial), and Contamination

Cell lines representing 15 different human and mouse cell types, respectively, are obtained from ATCC. For the studies of cell line identification and temporal divergence, each cell line is cultured according to the ATCC's recommended protocols and cells are harvested at a series of passages (1, 5, 10, 15, 20, 30, and 50). To investigate spatial divergence of cell lines, aliquots of the HEK 293 cells are obtained from at least three different laboratories and they are compared to the ATCC reference line without any further culturing. In addition, two types of biological contamination, which are relatively difficult to detect, are simulated in culture settings using either human or mouse cell lines purchased from ATCC: 1) cross-contamination by another cell line and 2) contamination with mycoplasma. Mycoplasma contamination can be confirmed by a commercial kit before landscape analysis.

Cells are harvested from individual experimental groups and snap-frozen. Genomic DNAs are isolated from the snap-frozen cell pellets, concentrations are measured, and their quality is evaluated by confirming the high molecular weight banding pattern prior to normalization to 20 ng/μl. The isolated genomic DNA samples is subjected to the HERV/MuERV landscape analyses.

### Collection of Heterogeneous HERV/MuERV Amplicons

Each human or mouse cell line has a dynamic and unique set of genomic TRE-landscapes which are formulated by the inherent diversity and acquired activity of ERVs (HERVs/MuERVs). These dynamic and heterogeneous genomic HERV/MuERV-landscapes, which are innate to each cell line, function as novel identifiers of the individual cell lines' temporal and spatial genomes.

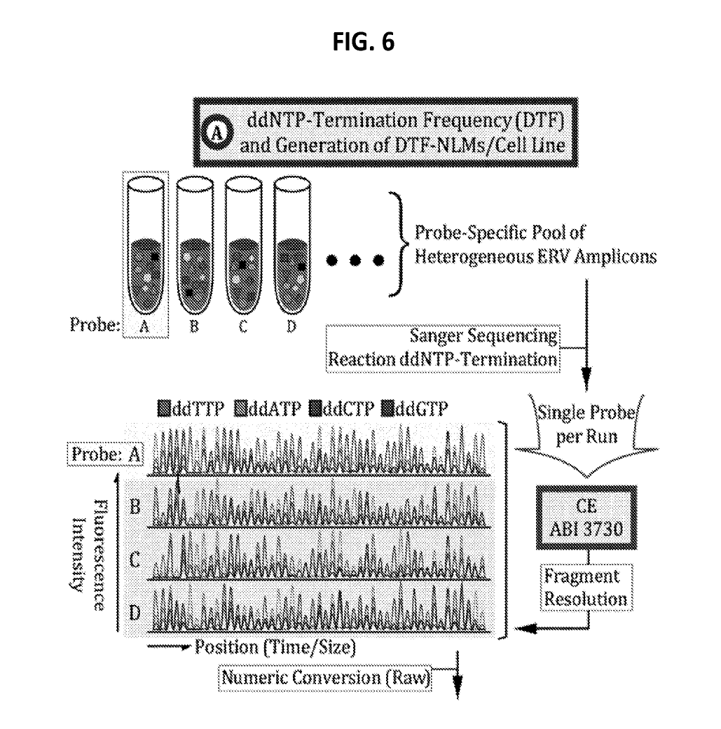

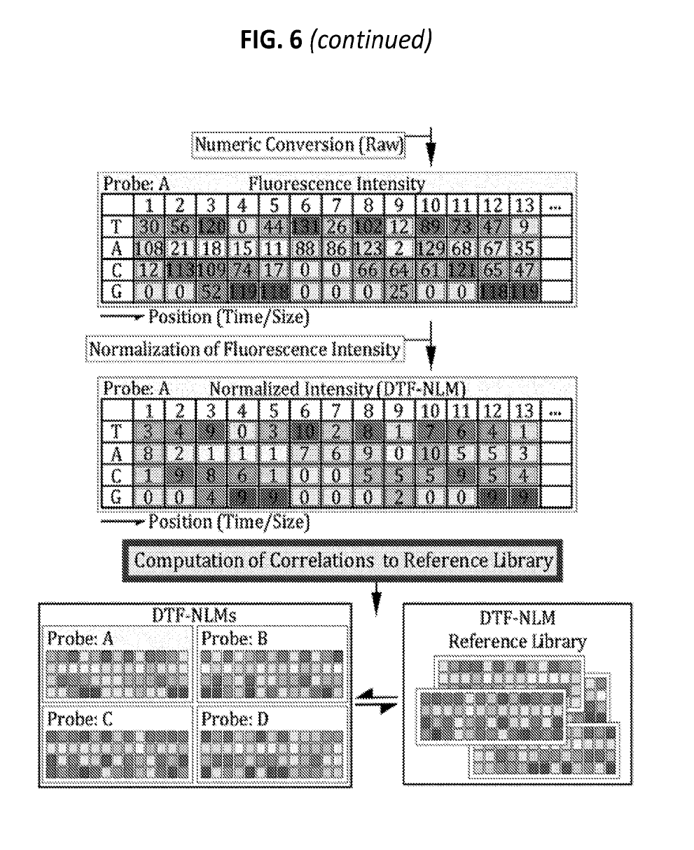

A population of heterogeneous HERVs/MuERVs (type and position), embedded in the genomes of individual cell lines, are obtained using HERV and MuERV landscaping probes (primer pairs) which are designed to PCR-amplify specific HERV/MuERV families and their insertion junctions/positions. DNA-processing protocols, such as restriction digestion and ligation, are used before or after PCR amplification (FIG. 6). The heterogeneous (size, sequence, and/or position) HERV/MuERV-landscape molecules for each cell line (including temporal, spatial, and contaminated ones) can be typically collected as: (1) a pool of HERV/MuERV-landscape amplicons derived from multiple PCRs (with or without digestion), (2) HERV/MuERV-landscape amplicons from single-multiplex PCR (with or without digestion), and (3) HERV/MuERV junction-landscape PCR amplicons (single or pool of multiple reactions) following digestion. The parameters for PCR and digestion are evaluated in order to render optimal resolution and/or size-spectrum of HERV/MuERV amplicons.

Numeric Transformation of HERV/MuERV Data by Dideoxynucleotide (ddNTP)-Termination

The HERV/MuERV-landscape amplicons are then subjected to the Sanger's ddNTP-termination reaction followed by resolution of nucleotide position-specific occurrence frequency of ddNTP-termination of individual nucleotides by running on four-color-fluorescent capillary electrophoresis (CE)-sequencing equipment, such as the ABI 3730 (FIG. 6). Each ddNTP type is labeled with a fluorescein of a unique wavelength. The ddNTP-termination reactions yield data with regard to the ddNTP-termination frequency (DTF) of individual nucleotides (A, C, G, or T) per nucleotide position, which is shared by the entire population of heterogeneous HERV/MuERV-landscape molecules. In contrast to the conventional Sanger sequencing data, which typically depicts one dominant fluorescence/peak at each nucleotide position, the DTF sequencing of a heterogeneous HERV/MuERV population generates a mosaic fluorescence pattern that represents the combination of A, C, G, and T at each position. The fluorescence intensity is directly converted to the DTF of the respective nucleotides at individual positions. The DTF values of a heterogeneous HERV/MuERV population, which are recorded as intensity of fluorescence with different wavelengths, are transformed into a matrix of numbers (fluorescence intensities) which consist of an X-Y plot of nucleotide position (variable) and type.

### Numeric Transformation of HERV/MuERV Data by Capillary Electrophoresis (CE)

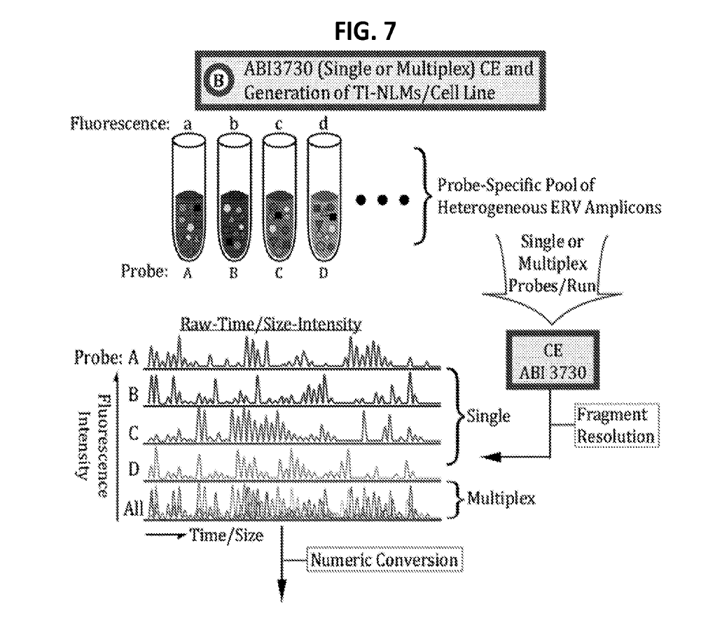

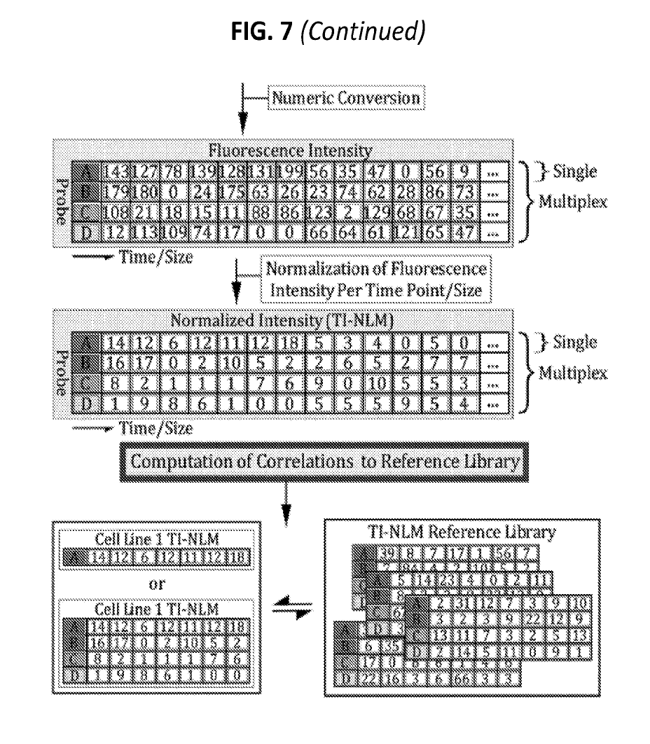

In addition to the ddNTP-termination strategy, the HERV/MuERV amplicons, are subjected to asymmetric PCR with the dominant primer labeled with a fluorescent dye which is specific for each HERV/MuERV subfamily/probe region in order to fluorescently label and amplify the landscape amplicons. Subsequently, the size and intensity profiles of the populations of heterogeneous HERV/MuERV-landscape amplicons are resolved by fluorescent CE using the ABI 3730 which can analyze four different fluorescent wavelengths (FIG. 7). On the other hand, conventional capillary electrophoretic separation yields thousands of time/size-intensity (TI) data points over a typical run period. For each wavelength, the outputs are recorded as an electropherogram or a raw numeric dataset of the amplicon intensity per read time point/size (e.g., every 0.2 seconds). In addition to the multi-fluorescent ABI 3730 system, other types of CE instruments (e.g., QIAxel or 2100 Bioanalyzer), which do not resolve multi fluorescence labels, can also be used. With these instruments, the HERV/MuERV amplicons can be digested with a set of restriction enzymes before being resolved in order to accomplish finer-resolution genome landscape identification. Various CE running parameters are tested to achieve optimal resolution and/or size-spectrum of the TI datasets. More than one HERV/MuERV subfamily/probe can be employed for cross-confirmation of identity, divergence, and contamination.

### Normalization of Numeric HERV/MuERV-Landscape Matrix

To prepare the numeric HERV/MuERV-landscape matrices for correlation computation, the numeric matrices of D as well as TI values are normalized (FIG. 6 and FIG. 7). With regard to the DTF datasets, the primary fluorescence intensity values of individual nucleotides per position are normalized by calculating the relative intensity of each nucleotide at each position. A normalized landscape matrix (DTF-NLM) that is unique for each cell line is now ready for the downstream correlation computation. On the other hand, the TI datasets are normalized by dividing the intensity values by the baseline number. This creates a TI-normalized landscape matrix (TI-NLM) for each cell line for correlation computation.

### Correlation Computation of DTF-NLMs and TI-NLMs

For cell line identification and surveillance, the DTF-NLMs or TI-NLMs from individual cell lines are subjected to correlation computation using a collection of established mathematical formulas: between two NLMs (contamination), among multiple NLMs (temporal and spatial divergence), or one NLM against a specific NLM library (identification). The correlation coefficient measures the strength of the relationship between two DTF-NLMs or TI-NLMs, which represent two genome samples. A value of zero indicates no relationship. A value of 1 indicates perfect positive correlation. For quantitative measurement of relationships among the genomes of a large and heterogeneous population of cell lines, the correlation coefficients of individual pairs are consolidated into a matrix for distance computation. To evaluate the accuracy and resolution of the NLM correlation values, a series of computation simulation studies can be performed using in silico-generated raw numeric HERV/MuERV-landscapes or NLMs of DTF- and TI-types. In addition, analytical protocols, which involve combinatorial interpretation of the NLM datasets (DTF- or TI-) obtained from two or more HERV/MuERV probes and/or restriction enzymes, are implemented in order to confirm identification and surveillance values.

### Construction of a Prototype Library of Cell Line-Specific DTF- and TI-NLMs

The DTF- and TI-NLMs of the total of 30 cell lines (human-15; mouse-15) analyzed in this example are organized into a prototype library of cell line-specific DTF- and TI-NLMs. Accumulation of HERV/MuERV-landscape matrices (DTF- and TI-NLMs) for a wide range of cell lines for each species leads to establishing machine-learnable NLM libraries which can be used for precise computation of identity, divergence, and contamination of cell lines.

### Expected Result's and Alternative Approach

This example refines the GST system for cell line authentication (with regard to identity, divergence, and contamination) and establishes a prototype library of HERV/MuERV-landscape DTF- and TI-NLMs for 30 cell lines of human and mouse origins. Together, the resources produced in this project can be the foundation of the projects which focus more on developing cell line authentication systems and relevant products. As an alternative for the DTF- and TI-based landscape analysis, the next generation sequencing (NGS) approach can be used for genome-wide HERV/MuERV position mapping. The NGS approach requires a tool which can efficiently capture the HERV/MuERV insertion-junctions embedded in the NGS read population. In addition, HERV/MuERV biochip systems, which are seeded with oligonucleotide probes representing the HERV/MuERV insertion positions annotated in the libraries, can be developed for a rapid mapping of HERV/MuERV positions for authentication of cell lines. The biochip systems can be updated as additional types and positions are annotated to the HERV/MuERV libraries, and can be customized for specific chromosomes and/or disease models.

Differential identification of cell lines based on the genomic TRE-landscaping technologies can significantly improve the confidence level of proper authentication. The probability of accurate identification of cell lines with regard to identity, divergence, and contamination is exponentially higher. Importantly, the current STR/gene polymorphism-based methods are not able to detect the divergence and contamination of cell lines primarily due to its inherently low resolution. For instance, implementing 32 HERV loci information derived from a single HERV probe reaction, instead of 16 STR loci (a current standard of cell line authentication) data, can decrease the likelihood of misidentification of cell lines by a factor of one billion (1×10⁹), using the assumption of independence and the multiplication rule. In fact, the described methods can generate at least a few dozen HERV/MuERV loci from a single probe (a pair of primers) reaction. Moreover, the extensive inherent and acquired polymorphisms in genomic TRE-type/position landscapes further can be used for differentiation of cell lines from gender-matching close relatives and monozygotic twins (humans) as well as gender-matching individual mice from an inbred strain. The probability of false positives will also decrease based on conditional probability when combined with other lines of information derived from independent probes and/or data transformation protocols.

### Example 5: Cell Line Authentication System

Within the GST system which is refined in Example 4, dynamic and high-resolution HERV/MuERV information from human and mouse cell lines is collected, numerically transformed, normalized, and correlation-computed to produce quantifiable and machine-learnable genetics surveillance values with regard to identity, divergence, and contamination.

### Development of HERV/MuERV-Landscaping Probe (Primer Pairs) Kits

In Example 4, two types of HERV/MuERV-landscaping probes (at least 100 for each species) are designed for: 1) probe regions on hyper-variable LTR regions for standard PCR (both unlabeled and fluorescently labeled) and 2) inverse-PCR (I-PCR) probe regions typically on LTRs (both unlabeled and fluorescently labeled). Efficacy of each probe for landscaping analysis, primarily with regard to the size- and population density-spectrums of amplicons derived from each probe, is evaluated in Example 4. The HERV/MuERV probes, including fluorescently labeled ones, which are determined to be efficient for high-resolution genome landscaping, are further selected for the production of primer kits for the authentication of cell lines of human and mouse origins. The oligonucleotide primers can be mass-synthesized, purified, packaged, and labeled.

During the production of HERV/MuERV-landscaping probe kits, quality control measures are implemented focusing on the following aspects: 1) DNAse- and RNAse-free conditions, 2) precise primer/oligonucleotide concentration, 3) confirmation of fluorescence-labeling chemistry, 4) signal-to-noise ratio of fluorescent labels, 5) precision dilution in specified buffers, 6) purity confirmation, 7) mixing of multiple primers, and 8) tracking of reagent source or batch/lot.

### Development of Programs for Capture, Numeric Transformation, Normalization, and Computation of HERV/MuERV-Landscape Datasets

The prototype computation algorithms, which are optimized and refined in Example 4, are developed into a suite of programs for capture, numeric transformation, normalization, and correlation computation of the HERV/MuERV-landscape datasets for cell line authentication. FIG. 8 illustrates the schema of the general Genetics Surveillance Systems, which include the quantitative and machine-learnable cell line authentication system. The quantitative and machine-learnable cell line authentication system can share the same schema.

The data capture and numeric transformation program can be designed to have specific data formats for each instrument (e.g., ABI 3730, QIAxel). The platform for this suite of programs can be built with standardized and open-source software in conjunction with leveraging the existing advancement of the field. In addition, cloud computing and storage can be implemented for an efficient deployment of the cell line authentication system and to facilitate collaborations. The cell line landscape reference databases for authentication, including contamination reference databases, are constructed.

Generation of DTF-NLM and TI-NLM Cell Line Reference Library of ˜125 Human and ˜75 Mouse Cell Types Obtained from ATCC

Using the GST-based genome landscaping systems, DTF-NLMs and TI-NLMs of ˜125 human and ˜75 mouse cell lines, which cover the significant majority of the ATCC-listed cell types, are produced at least with five probes (HERV or MuERV) per cell line for each species. This experiment can yield species-specific libraries of DTF/TI-NLMs which serve as a computable and machine-learnable reference library for cell line authentication with regard to identity and divergence (temporal and spatial).

### Generation of DTF-NLM and TI-NLM “Mycoplasma-Contaminated” Cell Line Reference Library of ˜125 Human and ˜75 Mouse Cell Types

Each of the ˜125 human and ˜75 mouse cell lines are contaminated with mycoplasma followed by generation of respective “contaminated” DTF-NLMs and TI-NLMs using at least five probes (HERV or MuERV) per cell line for each species. The outcomes are mycoplasma contamination-specific libraries of DTF/TI-NLMs which can serve as a reference for authentication of cell lines with regard to mycoplasma contamination. If a better resolution is needed for identifying contamination, one or two mycoplasma genome-specific probes are added when TRE-landscape amplicons are collected from the cell lines' genomes.

### Construction of “Cell Line Landscape Reference” (CLLR) Database Management System (DBMS)

To authenticate cell lines using the GST-landscaping system, the DTF/TI-NLM libraries of normal and “contaminated” cell lines are organized into the “Cell Line Landscape Reference (CLLR)” DBMS (FIG. 8). In addition, the DBMS can be equipped with the suite of programs for capturing, numeric transformation, normalization, and correlation computation of the HERV/MuERV-landscape datasets as well as user interfaces which allow for individual researchers or service providers to perform their cell line authentication on-line.

### Expected Results and Alternate Approach

It is expected that a cell line authentication database can be built by the methods described herein. Additional HERV/MuERV probes which can be used to collect genomic landscape elements for specifically identifying/confirming the original tissue types/cell types of individual cell lines are identified. In addition to the two species (human and mouse), the CLLR DBMS can be expanded to other species.

An alternative strategy for this quantitative genome-landscaping based cell line authentication would involve resolution of the heterogeneous HERV/MuERV-landscape amplicons from single or mixed fluorescent (optional) probes on long-range polyacrylamide gels. In this qualitative approach, a library of visual banding patterns of HERV/MuERV landscapes, which specifically identify individual cell lines, can be established as an authentication reference database within each species. One advantage of this visual approach is that individual research laboratories can analyze the HERV/MuERV-landscape amplicons, which are produced using the probe kits developed for the quantitative system, and authenticate their cell lines by querying the banding patterns directly to the respective visual reference databases.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of creating a dideoxynucleotide termination frequency (DTF) normalized landscape matrix or a time/intensity (TI) normalized landscape matrix, the method comprising:
(1) providing a plurality of amplicons having different genomic elements/sequences, optionally wherein the amplicons are provided by digestion and/or ligation of genomic DNA prior to PCR amplification;
performing a dideoxynucleotide termination sequencing reaction on a reaction mixture comprising the plurality of amplicons having different genomic elements/sequences, using a primer that binds to the plurality of amplicons at a plurality of different binding sites;
obtaining an intensity of fluorescence for each type of nucleotide (A, T, G, C) at each individual nucleotide position in the heterogeneous population of amplicons;
normalizing the intensity of fluorescence of each nucleotide type at each individual nucleotide positions;
creating a matrix of the normalized intensity of fluorescence for each type of nucleotide at each individual nucleotide position;
thereby creating a DTF normalized landscape matrix; or
(2) providing a plurality of amplicons having different genomic elements/sequences, optionally wherein the amplicons are provided by digestion and/or ligation of genomic DNA prior to PCR amplification;
performing capillary electrophoresis (CE) analysis of the plurality of amplicons having different sequences, optionally after restriction digestion;
obtaining time (second)/size-intensity (mV) values over a specified time period from the CE analysis;
normalizing the amplicon/fragment intensity at each time point/size by dividing the intensity values by a baseline value, thereby creating a normalized time/size-intensity landscape matrix (TI-NLM) for each sample;
thereby creating a TI normalized landscape matrix.

2. (canceled)

3. The method of claim 1, wherein the plurality of amplicons is obtained using one or more PCR reactions, wherein the PCR reactions are configured to amplify heterogeneous elements/regions in a genome.

4. The method of claim 1, wherein the plurality of amplicons is obtained using single-multiplex PCR.

5. The method of claim 1, wherein the plurality of amplicons comprise repetitive elements, B-cell receptors, T-cell receptors, or protocadherin gene clusters.

6. A method of determining a genetic identity of a cell, tissue, organ, or organism, the method comprising:
(1) creating a DTF or TI normalized landscape matrix for the genome of the cell, tissue, organ, or organism, according to the method of claim 1; and
(2) determining the distance-correlation between the DTF or TI normalized landscape matrix of a test sample and a DTF or TI normalized landscape matrix of a reference sample, optionally wherein the reference sample has a known genetic identity; and
(3) optionally determining whether the distance is less than a reference threshold;
thereby determining the genetic identity of a cell, tissue, organ, or organism.

7. The method of claim 6, wherein the cell, tissue, organ, or organism is, or is from, an animal, a plant, a fungus or a bacterium.

8. The method of claim 7, wherein the animal is a mammal (e.g., a human), a bird, a fish, or a reptile.

9. The method of claim 6, wherein the cell, tissue, organ, or organism is, or is from, a genetically modified animal or a genetically modified plant.

10. A method of determining whether a test subject has a disease, the method comprising:
a) creating a DTF or TI normalized landscape matrix of the test subject according to the method of claim 1;
b) calculating the distance between the DTF or TI normalized landscape matrix of the test subject and one or more DTF or TI normalized landscape matrices that represent a subject having the disease; and
c) comparing the distance to a reference threshold, and concluding that the test subject has the disease if the distance is less than a reference threshold.

11. The method of claim 10, wherein the disease is cerebral palsy, autism spectrum disorder, ductal carcinoma in situ, breast cancer or an aging-related disorder.

12. A method of identifying a genetic risk factor in a test subject, the method comprising:
a) creating a DTF or TI normalized landscape matrix of the test subject according to the method of claim 1;
b) calculating the distance between the DTF or TI normalized landscape matrix of the test subject and one or more DTF or TI normalized landscape matrices representing a subject having the genetic risk factor; and
c) comparing the distance to a reference threshold, and identifying the test subject as having the genetic risk factor if the distance is less than a reference threshold.

13. The method of claim 12, wherein the test subject is a fetus or an embryo.

14. A method of monitoring a genome of a subject, the method comprising:
a) creating a DTF or TI normalized landscape matrix for the subject at a first time point according to the method of claim 1;
b) creating a DTF or TI normalized landscape matrix for the subject at a second time point; and
c) calculating the distance between the DTF or TI normalized landscape matrix of the first time point and the DTF or TI normalized landscape matrix of the second time point;
thereby monitoring the genome of the subject.

15. The method of claim 15, wherein the subject is receiving a therapy between the first and second time points.


SEQUENCE LISTING (2 sequences)

SEQ ID NO: 1 - DNA, 20 bp, Artificial Sequence
  synthetic primer
aggcaagagactgaaggcac

SEQ ID NO: 2 - DNA, 18 bp, Artificial Sequence
  synthetic primer
gtagggctggaccctaca